(19) United States
(12) Patent Application Publication
(10) Pub. No.: US 2007/0274963 A1
(43) Pub. Date: Nov. 29, 2007

(54) Methods for Culturing Keratinocytes from Human Embryonic Stem Cells

(75) Inventors: Howard Green, Brookline, MA (US); Shiro Iuchi, Quincy, MA (US); Karen Easley, Tewksbury, MA (US)

Correspondence Address: WOLF GREENFIELD & SACKS, P.C., 600 ATLANTIC AVENUE, BOSTON, MA, 02210-2206, US

(73) Assignee: President and Fellows of Harvard College, Cambridge, MA (US)

(21) Appl. No.: 10/582,028

(22) PCT Filed: Dec. 8, 2004
(86) PCT No.: PCT/US04/40977

§ 371 Date: Mar. 19, 2007

Related U.S. Application Data
(60) Provisional application No. 60/527,920, filed on Dec. 8, 2003.

(51) Int. Cl. C12N 5/06; C12N 5/08

(52) U.S. Cl. 424/93.7; 435/29; 435/371; 435/378

(57) ABSTRACT

The invention relates to methods for isolating and culturing human keratinocytes from embryonic stem cells. The methods are useful for producing substantially pure cultures of keratinocytes.

### RELATED APPLICATION

This application claims priority under 35 U.S.C. § 119 from U.S. provisional application Ser. No. 60/527,920, filed Dec. 8, 2003, the contents of which is incorporated herein in its entirety.

### GOVERNMENT SUPPORT

This invention was made in part with government support under grant number R01GM068478-01 from the National Institutes of Health (NIH). The government may have certain rights in this invention.

### FIELD OF THE INVENTION

The invention relates to methods of isolating and culturing human keratinocytes from embryonic stem cells. The methods are useful for producing substantially pure cultures of keratinocytes.

### BACKGROUND OF THE INVENTION

The ability to culture keratinocytes has enabled advancements in the treatment of burns and trauma that affect the skin. The likelihood of survival of patients with severe burns has improved dramatically with the advent of methods to replace lost skin with laboratory-grown skin cells. One source of replacement skin tissue is through the use of cell culture methods to produce sheets of keratinocytes. Embryonic stem (ES) cells have been identified as a potential source for keratinocytes, but efficient strategies to identify ES cells that will differentiate into keratinocytes are not available, and methods to isolate and culture ES cells of keratinocyte lineage are also lacking.

Human embryos can for obvious reasons not be used experimentally to study early development. Embryos of other mammalian species, especially the mouse, have been used a great deal and much has been learned from their study. But the increasing structural complexity of the embryo after implantation imposes great difficulties for the study of the consecutive changes that lead from stem cells to definitive somatic cell types.

Embryonic stem (ES) cells of the mouse, first grown in cell culture by Evans and Kaufman (Evans, M. J. & Kaufman, M. H., Nature 292: 154-156, 1981.) and Martin (Martin, G. R., Proc. Natl. Acad. Sci. USA 78: 7634-7638, 1981.) give rise to many forms of differentiation (Smith, A. G., Annu. Rev. Cell Dev. Biol. 17: 435-462, 2001.). One such example, from the laboratory of F. Watt, is the formation of keratin-containing cells from murine ES cells (Bagutti, C. et al., Dev. Biol. 179:184-196, 1996; Bagutti, C. et al., Dev. Biol. 231: 321-333, 2001.). Later, more advanced differentiation of keratinocytes was obtained (Coraux, C. et al., Curr. Biol. 13: 849-853, 2003.), but no definitive keratinocytes were isolated in these studies even though keratinocytes of a murine teratoma had earlier been serially cultivated (Rheinwald, J. G. & Green, H., Cell 6: 317-330, 1975.). One reason may be that, on serial cultivation, rodent somatic cells tend to convert quickly into established (“immortal”) cell lines with altered properties.

Human ES cells were first cultivated by Thomson and coworkers (Thomson, J. A. et al., Science 282: 1145-1147, 1998.) and, like murine ES cells (Robertson, E. J. in Teratocarcinomas and Embryonic Stem Cells: A Practical Approach, 1987.), they differentiate into many cell types (Thomson, J. A. et al., Science 282: 1145-1147, 1998; Thomson, J. A. & Odorico, J. S., Trends Biotechnol. 18: 53-57, 2000; Reubinoff, B. E. et al., Nat. Biotechnol. 18: 399-404, 2000; Itskovitz-Eldor, J. et al., Mol. Med. 6: 88-95, 2000.), but unlike murine somatic cells, human somatic cells tend to be stable in culture and rarely develop spontaneously into established cell lines. Methods of influencing or directing differentiation to certain somatic cell types have been described for murine ES cells (Lumelsky, N. et al., Science 292: 1389-1394, 2001.), for human ES cells (Schuldiner, M. et al., Proc. Natl. Acad. Sci. USA 97: 11307-11312,2000; Schuldiner, M. et al., Brain Res. 913: 201-205, 2001.), for human embryonic germ cells (Shamblott, M. J. et al., Proc. Natl. Acad. Sci. USA 98: 113-118, 2001.), and for developing embryos (Kumar, M. & Melton, D., Curr. Opin. Genet. Dev. 13: 401-407, 2003.). When transplanted to scid mice, human ES cells have been shown to give rise to respiratory and gut epithelium, bone, cartilage, smooth and striated muscle, ganglia, renal structures, and stratified squamous epithelium with hair follicles (Thomson, J. A. & Odorico, J. S., Trends Biotechnol. 18: 53-57, 2000; Reubinoff, B. E. et al., Nat. Biotechnol. 18: 399-404, 2000; Thomson, J. A. et al., Proc. Natl. Acad. Sci. USA 92: 7844-7848, 1995.). This finding demonstrates that keratinocytes are generated from human ES cells in the absence of embryonic implantation and the orderly sequence of fetal development.

A drawback of these methods is the lack of accessibility of the differentiating cells for examination. The current methods for identifying and culturing keratinocytes are limited in that they do not allow identification of human ES cells of keratinocyte lineage early in the developmental process. Also, present methods utilize incomplete selections of markers and result in cells that are mixed, heterogeneous cultures, not isolated single cell types. In addition, current methods are not sufficient to allow reliable and efficient isolation and growth of human ES cells that will differentiate and may stratify as keratinocytes under suitable culture conditions. Thus, a need exists for efficient and reproducible methods for isolating and culturing human keratinocytes from human ES cells.

### SUMMARY OF THE INVENTION

We have discovered novel methods of identifying, isolating, and culturing keratinocytes from human embryonic stem cells. The methods of the invention allow the production of sheets of human keratinocytes, which can be used for the treatment of burns and trauma, and also can be used as experimental compositions and substrates.

The methods of the invention are advantageous in that they allow the analysis of the ES cell differentiation process in an accessible system. By growing ES cells in culture, the differentiating cells are accessible and can be isolated for further expansion. We have discovered methods of harvesting ES cell-derived keratinocytes from ES cell nodules. The methods of the invention also include, in part, contacting the harvested cells from the ES cell nodule with low-Ca⁺⁺ medium to selectively eliminate ES cells from the culture, and their subsequent multiplication permitting the production of substantially pure ES cell-derived keratinocyte cultures from ES cell nodules.

ES cells may also be grown in culture under conditions in which migrating and differentiating cells originating from ES cells have an essentially 2D (“monolayer”) structure. We have examined the cells in this monolayer structure, and have standardized the starting conditions and stages of keratinocyte differentiation. Thus, we have developed methods for producing stable human keratinocytes from ES cells. The keratinocytes produced using the methods of the invention can be used for treatment of burns and/or trauma that necessitates the replacement of human skin. We have also developed a two-stage culture method for prolonging the growth of ES cell-derived keratinocytes. In one stage, ES-derived keratinocytes are cultured in low-Ca⁺⁺ medium for one or more passages. In a subsequent stage, the cells are cultured in a suitable culture medium for keratinocyte growth such as cFAD.

In addition, we have developed methods of producing a mega-embryoid body that is useful for obtaining embryonic stem cells. The mega-embryoid bodies prepared using the methods of the invention are larger than standard embryoid bodies prepared using conventional methods. In addition the mega-embryoid bodies allow faster harvesting of keratinocytes than does use of standard embryoid bodies in keratinocyte harvest methods.

According to one aspect of the invention, methods of making a substantially pure culture of embryonic stem (ES) cell-derived keratinocytes is provided. The methods include expanding selectively a keratinocyte derived from cultured ES cells to obtain a substantially pure culture of ES cell-derived keratinocytes. In one embodiment, a cell from an aggregate that is an ES cell nodule can be selectively expanded in culture. In some embodiments, a keratinocyte from an ES cell nodule can be expanded selectively in culture with low-Ca⁺⁺ medium. In other embodiments, ES cells of an aggregate that is an embryoid body or a mega-embryoid body can be expanded selectively in culture. A variety of methods can be used to expand selectively a keratinocyte derived from cultured ES cells, two of which are described in more detail below.

According to another aspect of the invention, methods of making a substantially pure culture of ES cell-derived keratinocytes are provided. The methods include expanding a keratinocyte cell harvested from an ES cell nodule to obtain a substantially pure culture of ES cell-derived keratinocytes. In some embodiments, the cells harvested from the ES cell nodule are contacted with low Ca⁺⁺ medium to selectively deplete ES cells from the harvested cells. In certain embodiments, the embryonic stem (ES) cell nodule is a human ES cell nodule. In some embodiments, the embryonic stem (ES) cell nodule is prepared in a scid mouse. In certain embodiments, the means of harvesting the keratinocyte cell from the ES cell nodule comprises disaggregation of the ES cell nodule. In some embodiments, the disaggregation of the ES cell nodule includes contacting the ES cell nodule with trypsin. In some embodiments, the harvested keratinocyte cell is expanded in low Ca⁺⁺ medium with or without 3T3 cells or other strain of embryonic fibroblast. In some embodiments, the harvested keratinocyte cell is expanded in cFAD medium with or without 3T3 cells or other strain of embryonic fibroblast. In some embodiments, the 3T3 cells or other strain of embryonic fibroblast are irradiated cells. In some embodiments, the keratinocyte cell is first expanded for one or more passages in low-Ca⁺⁺ medium with or without 3T3 cells or other strain of embryonic fibroblasts and subsequently expanded for one or more passages in cFAD medium with or without 3T3 cells or other strain of embryonic fibroblast. In certain embodiments, the low Ca⁺⁺ medium is serum-free medium. In some embodiments, the cFAD medium comprises 10% (v/v) fetal calf serum. In some embodiments, the cells of keratinocyte lineage are cells that display one or more markers selected from the group consisting of: p63, K14, basonuclin, involucrin, colony fragmentation, and circumferential movement. In some embodiments, the methods also include administering keratinocytes from the substantially pure culture of ES cell-derived keratinocytes to a subject for the treatment of a wound. In some embodiments, the method also includes administering a composition that includes keratinocytes from the substantially pure culture of ES cell-derived keratinocytes to a subject for the treatment of a wound.

According to another aspect of the invention, products are provided. The products can be formed by any of the foregoing methods of the invention.

According to yet another aspect of the invention, methods of making a substantially pure culture of ES cell-derived keratinocytes are provided. The methods include culturing embryonic stem cells and expanding the number of cells of the keratinocyte lineage derived from the ES cells to obtain a substantially pure culture of ES cell-derived keratinocytes. In some embodiments, the embryonic stem cells are an aggregate. In certain embodiments, the aggregate comprises two or more human embryonic stem cells. In some embodiments, the aggregate is a human embryoid body or a mega-embryoid body. In certain embodiments, the aggregate is cultured on a surface adapted for cell attachment, for a time sufficient to permit cells to grow and migrate distally from the aggregate. In some embodiments, the cells that migrate distally away from the cultured aggregate are cells of keratinocyte lineage. In some embodiments, the surface adapted for cell attachment is a cell culture dish. In certain embodiments, the time sufficient to permit cells to grow and migrate distally from the human embryoid body is at least about 10 days. In some embodiments, the cells are permitted to grow and migrate distally from the human embryoid body for about 10, 11, 12, 13, 14, 15, 16, 17, 18, 19, 20, 21, 22, 23, 24, or 25 days. In some embodiments, the time sufficient to permit cells to grow and migrate distally from the mega-EB is at least about 1 day. In certain embodiments, the cells are permitted to grow and migrate distally from the mega-EB for at least about 1, 2, 3, 4, 5, 6, 7, 8, 9, 10, 11, 12, 13, 14, or 15 days. In some embodiments, the aggregates are cultured in cFAD medium on irradiated 3T3 cells or other strain of embryonic fibroblast. In some embodiments, the cells of keratinocyte lineage are expanded in serum-free medium with or without irradiated 3T3 cells or other strain of embryonic fibroblast. In certain embodiments, the cells of keratinocyte lineage are first expanded for one or more passages in low-Ca⁺⁺ serum-free medium with or without 3T3 cells or other strain of embryonic fibroblast and subsequently expanded for one or more passages in cFAD medium with or without 3T3 cells or other strain of embryonic fibroblast. In some embodiments, the cFAD medium comprises 10% (v/v) fetal calf serum. In some embodiments, the 3T3 cells or other strain of embryonic fibroblast are irradiated cells. In certain embodiments, the cells of keratinocyte lineage are cells that display one or more markers selected from the group consisting of: p63, K14, basonuclin, involucrin, colony fragmentation, and circumferential movement. In some embodiments, the methods also include administering keratinocytes from the substantially pure culture of ES cell-derived keratinocytes to a subject for the treatment of a wound. In some embodiments, the method also includes administering a composition that includes keratinocytes from the substantially pure culture of ES cell-derived keratinocytes to a subject for the treatment of a wound. In some embodiments, a product formed by the any of the forgoing methods of the invention is provided.

According to yet another aspect of the invention, methods of treating a skin injury in a subject are provided. The methods include administering to a subject in need of such treatment an ES cell-derived keratinocyte made with the method of any of the foregoing aspects of the invention in an amount effective to treat the skin injury. In some embodiments, the skin injury is the result of disease or trauma. In some embodiments, the trauma is a burn.

According to yet another aspect of the invention, methods of treating a skin injury in a subject are provided. The methods include obtaining an ES cell-derived keratinocyte made with the method of any of the foregoing aspects of the invention cell and administering the ES cell-derived keratinocyte to a subject in need of such treatment in an amount effective to treat the skin injury. In some embodiments, the skin injury is the result of disease or trauma. In some embodiments, the trauma is a bum.

According to another aspect of the invention, methods of identifying an ES cell-derived cell for treating an injury in a subject are provided. In some embodiments, the methods include contacting an ES cell-derived cell in culture with retinoic acid, determining the presence of circumferential movement in the contacted cell, wherein the presence of circumferential movement identifies the cell for treating injury in the subject. In some embodiments, the retinoic acid is at a concentration in the culture of between about 10⁻⁷ molar and 10⁻¹⁰ molar. In some embodiments, the retinoic acid is at a concentration in the culture of about 10⁻⁷ molar. In certain embodiments, the ES cell-derived cell is an ES cell-derived keratinocyte. In some embodiments, the ES cell-derived keratinocyte is an ES cell-derived keratinocyte made with the method of the foregoing aspects of the invention.

According to yet another aspect of the invention, methods of identifying an ES cell-derived keratinocyte for treating an injury in a subject are provided. The methods include culturing an ES cell-derived cell, wherein the cell forms a colony, determining the presence of fragmentation of the colony, wherein the presence of the fragmentation identifies the keratinocyte for treating injury in the subject. In some embodiments, the ES cell-derived cell is a cell from an ES cell nodule. In certain embodiments, the ES cell-derived keratinocyte is an ES cell-derived keratinocyte made with the methods of any of the foregoing aspects of the invention.

According to yet another aspect of the invention, methods of preparing a mega-embryoid body are provided. The methods include (a) expanding ES cells in a culture that includes culture medium and fibroblasts, (b) recovering the expanded cells, (c) growing the recovered cells in an inverted vessel that includes culture medium, and (d) culturing the cells upright for at least one additional day, wherein the cultured cells form a mega-embryoid body. In some embodiments, the culture medium is SR medium. In certain embodiments, the recovered cells are grown in the inverted vessel for about 1, 2, 3, or 4 days. In some embodiments, the method also includes culturing human keratinocytes from the mega embryoid body (mega-EB) on a surface in attachment culture medium. In some embodiments, the mega-EB is cultured for at least about 24 hours. In some embodiments, the attachment culture medium is cFAD medium. In certain embodiments, the methods also include culturing human ES cell-derived keratinocytes from the mega embryoid body (mega-EB) using methods of any of the foregoing aspects of the invention.

According to another aspect of the invention, methods of preparing a mega-embryoid body are provided. The methods include expanding ES cells in an inverted vessel that includes culture medium, and culturing the cells upright for at least one additional day, wherein the cultured cells form a mega-embryoid body. In some embodiments, the culture medium is SR medium. In certain embodiments, the recovered cells are grown in the inverted vessel for about 1, 2, 3, or 4 days. In some embodiments, the method also includes culturing human keratinocytes from the mega embryoid body (mega-EB) on a surface in attachment culture medium. In some embodiments, the mega-EB is cultured for at least about 24 hours. In some embodiments, the attachment culture medium is cFAD medium. In certain embodiments, the methods also include culturing human ES cell-derived keratinocytes from the mega embryoid body (mega-EB) using methods of any of the foregoing aspects of the invention.

According to yet another aspect of the invention, methods of identifying an ES cell-derived keratinocyte are provided. The methods include contacting an ES cell-derived cell in culture with retinoic acid, determining the presence of circumferential movement in the contacted cell, wherein the presence of circumferential movement identifies the cell as an ES cell-derived keratinocyte. In some embodiments, the retinoic acid is at a concentration in the culture of about 10⁻⁷ to 10⁻¹⁰ molar. In some embodiments the retinoic acid is at a concentration in the culture of about 10⁻⁷ molar. In certain embodiments, the methods also include expanding the identified keratinocyte under conditions to permit colonization of a substantially pure culture of keratinocytes. In some embodiments, the methods also include using one or more keratinocytes from the substantially pure culture of keratinocytes to treat an injury in a subject.

According to yet another aspect of the invention, methods of treating a skin injury in a subject are provided. The methods include administering to a subject in need of such treatment an ES cell-derived keratinocyte identified with the methods of any of the foregoing claims in an amount sufficient to treat the skin injury. In some embodiments, the skin injury is the result of disease or trauma. In certain embodiments, the trauma is a burn.

According to yet another aspect of the invention, compositions are provided. The compositions include an embryonic stem cell-derived keratinocyte made with the method of any of the foregoing aspects of the invention or identified with a method of any of the foregoing aspects of the invention.

According to another aspect of the invention, products formed by any of the foregoing aspects of the invention are provided.

The use of the foregoing ES cell-derived keratinocytes in the preparation of a medicament, particularly a medicament for treatment of skin injury or disorder, including but not limited to burns, trauma, and disease is also provided.

These and other objects of the invention will be described in further detail in connection with the detailed description of the invention.

Each of the limitations of the invention can encompass various embodiments of the invention. It is, therefore, anticipated that each of the limitations of the invention involving any one element or combination of elements can be included in each aspect of the invention.

This invention is not limited in its application to the details of construction an the arrangement of components set forth in the following description or illustrated in the drawings. The invention is capable of other embodiments and of being practiced or o being carried out in various ways.

### BRIEF DESCRIPTION OF THE DRAWINGS

FIG. 1 is a diagram illustrating marker succession in the keratinocyte lineage.

FIG. 2 shows digitized images of colonies formed in primary culture of an ES cell-produced nodule in a scid mouse. FIG. 2A shows a keratinocyte colony 5 days after inoculation of disaggregated cells of the nodule. FIG. 2B shows the same colony 7 days after inoculation. The number of cells has increased from 127 to 685, corresponding to a T_{d} of 21 hours.

FIG. 3 shows digitized images of post-natal keratinocytes in culture demonstrating colony morphology. The colonies are 8-day colonies formed by foreskin keratinocytes, strain YF29, passage VI, following their inoculation into a dish containing 3T3 support. Most colonies approach circularity of outline. All colonies are coherent and expand by excavating neighboring 3T3 cells from the vessel surface. (Phase contrast, 4× objective).

FIG. 4 shows digitized images of four samples of ES-derived keratinocytes. Keratinocytes derived from nodules in scid mice were serially transferred with 3T3 support. All photographs (FIGS. 4A-D) show colonies 8-10 days after plating of passage VII. (Phase contrast, 20× objective). These colonies are much small than those of post-natal keratinocytes at the same time after inoculation. The colonies are irregular in outline and appear to be breaking up by movement of parts of the colony in opposite directions (to left or to right).

FIG. 5 shows digitized images of keratinocyte colony formed from an attached embryoid body of 0.7 mm in length. FIG. 5A shows part of a colony, of a total size 5 mm×5 mm and containing over 7000 cells, after 26 days of migration of cells from the attached EB and their subsequent multiplication. (phase contrast, 4× objective). FIG. 5B shows a higher power view of the expanding edge of the colony 6 days earlier showing cells of rather homogeneous appearance typical of keratinocyte. (phase contrast, 20× objective).

### DETAILED DESCRIPTION OF THE INVENTION

The invention disclosed herein describes novel methods of identifying, isolating, and culturing keratinocytes from human embryonic stem cells. The discovery that the stages and timing of differentiation of ES cells into keratinocytes can be monitored in culture facilitates the production of keratinocyte tissue, including sheets of keratinocytes, for use in the treatment of burns and/or trauma to the skin. In addition, we have also identified a set of markers, including transcription markers and differentiation markers that are useful in the identification of cells of keratinocyte lineage isolated from ES cells.

The methods of the invention relate to the isolation and culture of keratinocytes from embryonic stem (ES) cells. As used herein the term “embryonic stem (ES) cells” means mammalian embryonic stem cells. As used herein the term “ES cell-derived keratinocytes” means keratinocytes that have been derived from embryonic stem cells. Embryonic stem cells are pluripotent cells that are derived from pre-implantation embryos. ES cells have the capacity to differentiate into any cell type in vivo, and to differentiate into many different cell types in vitro.

One cell type that may arise from the differentiation of ES cells in vivo or in vitro is a keratinocyte. As used herein, a cell of “keratinocyte lineage” is a cell that differentiates from an ES cell to a keratinocyte under suitable growth conditions. Thus, a cell of keratinocyte lineage is a cell committed to be a keratinocyte. The invention, in part, involves use of methods to produce a substantially pure culture of keratinocytes. As used herein, the word “substantially pure culture” means cells grown in culture that are substantially free of other cultured cell types. In some embodiments, a substantially pure culture of keratinocytes may be grown on a support layer of cells (e.g. 3T3 cells). Support layer cells, which are also known as “feeder cells”, can be mitotically inactivated embryonic fibroblast cells, examples of which are irradiated 3T3 cells or other strain of embryonic fibroblast. It will be understood by those of skill in the art, that the mitotically inactivated embryonic cells, e.g. irradiated cells, on which the keratinocytes are grown are incapable of proliferation does not negate the fact that a culture of growing keratinocytes is substantially pure if at least 85%, 90%, 95%, 96%, 97%, 98%, 99%, or 100% of the growing cells in the culture are keratinocytes. The presence of only a small percentage or zero percentage of other growing cell types, including ES cells, in a culture of keratinocytes means the culture is a substantially pure culture of keratinocytes.

The ES cells of the invention can be ES cells obtained from any mammalian species including humans, non-human primates, cats, dogs, sheep, pigs, horses, cows, and rodents such as mice, rats, etc. In important embodiments, the ES cells used in the methods of the invention are human ES cells.

The ES cells of the invention may be cells that are part of ES cell aggregates. As used herein, the term “aggregate” means a group or cluster comprising at least two or more ES cells. ES cell aggregates as used in the methods of the invention, may be clusters or groups of ES cells. ES stem cells for use in the methods of the invention may be obtained directly from a mammalian pre-implantation embryo, or may be cultured ES stem cells. Examples of ES cell aggregates, although not intended to be limiting, include ES cell nodules, embryoid bodies, and mega-embryoid bodies.

ES cell nodules are routinely used in the art and methods of procuring and maintaining ES cell nodules are known to those of ordinary skill in the art. For example, as described in the Examples section, injecting ES cells into scid mice results in the formation of nodules. ES cell nodules comprise ES cells and cells derived from them, including keratinocytes. Thus, in some embodiments, keratinocytes are cultured in vivo as part of an ES cell nodule. In some embodiments of the invention the ES cells injected into the scid mouse are human ES cells.

The invention, in part, includes methods for producing a substantially pure culture of ES cell-derived keratinocytes from cells harvested from an ES cell nodule. The methods include harvesting keratinocytes from an ES cell nodule. Keratinocytes can be harvested by disaggregating the ES cell nodule. Methods to disaggregate (dissociate) the cells of the ES cell nodule include, but are not limited to, contacting the ES cell nodule with an enzyme such as trypsin using art-known methods of cell disaggregation. In some embodiments of the invention, the cells harvested from the ES cell nodule are contacted with low Ca⁺⁺ medium to selectively deplete ES cells from the harvested cells.

As described herein, ES cell nodules include, but are not limited to ES cells and ES cell-derived keratinocytes. Contacting the cells disaggregated from the ES cell nodule with low Ca⁺⁺ medium selectively reduces the number of ES cells in the culture, while maintaining the number of keratinocytes in the culture. Thus, a mixture of ES and keratinocytes from a disaggregated nodule can be contacted with low Ca⁺⁺ medium and result in a substantially pure culture of keratinocytes. In some embodiments of the invention, the ES cell nodule is a human ES cell nodule that has been prepared in a scid mouse. Methods for preparing ES cell nodules in scid mice are known in the art.

After harvest, a keratinocyte from an ES cell nodule can be expanded in low Ca⁺⁺ medium with or without 3T3 cells or other strain of embryonic fibroblast. In some embodiments, the low-Ca⁺⁺ medium is serum-free medium. In some embodiments, the harvested keratinocyte cell can be expanded in cFAD medium with or without 3T3 cells or other strain of embryonic fibroblast. In some embodiments of the invention, cFAD medium contains fetal calf serum (FCS) in an amount ranging from about 5% up to about 15% (v/v) FCS. In some embodiments, the cFAD medium contains 10% (v/v) FCS. Additional conditions that are useful to permit colonization of keratinocytes from ES cell nodules include first culturing ES cell nodule harvested keratinocytes for one or more passages in low-Ca⁺⁺ serum-free medium with or without 3T3 cells or other strain of embryonic fibroblast and subsequently culturing the ES cell-derived keratinocytes for one or more passages in cFAD medium with or without 3T3 cells or other strain of embryonic fibroblast. In some embodiments of the invention, the cFAD medium also includes 10% (v/v) FCS. In some embodiments, the low Ca⁺⁺ medium is serum-free medium. It is also possible to determine the presence of various keratinocyte markers in cells from ES cell nodules to identify cells as keratinocytes. Cells that have one or more markers selected from the group consisting of: p63, K14, basonuclin, involucrin, colony fragmentation, and circumferential movement are identified as keratinocytes.

Keratinocytes derived from ES cell nodules can be used to make products. The products are useful for research methods and for methods of treating a skin wound on a subject. Keratinocytes from ES cell nodules can also be administered to a subject for the treatment of a wound.

Embryoid bodies are ES cell aggregates formed in vitro that are useful in the methods of the invention. Embryoid bodies are three-dimensional groups of ES cells and may include up to several thousand cells aggregated together. Embryoid bodies are routinely used in the art. Methods of procuring and maintaining embryoid bodies will be understood by those of ordinary skill in the art. In addition to art-known embryoid bodies, the invention also relates, in part, to the preparation and use of mega-embryoid bodies. Mega-embryoid bodies (mega-EBs), which are also referred to herein as multilocular embryoid bodies, can be made using methods provided herein (see Example 3). Mega-EBs can be used in methods to harvest keratinocytes, including, but not limited to, the keratinocyte-harvest methods described herein. Mega-EBs are larger than EBs that are prepared using conventional methods. ES cell-derived keratinocytes prepared from mega-EBs are ready for harvest in a shorter period of time than ES cell-derived keratinocytes prepared from regular embryoid bodies. Thus, the use of mega-EBs can reduce the time required for harvesting ES cell-derived keratinocytes.

The methods of the invention include culturing one or more ES cell aggregates under conditions to permit the growth and/or migration of from the aggregate of cells of the keratinocyte lineage. In some embodiments, an ES cell aggregate may be placed on a surface adapted for cell attachment. As used herein, the term “adapted for cell attachment” includes surfaces on which the aggregate will adhere. Examples of surfaces that are adapted for cell attachment include, but are not limited to standard tissue culture plates, tubes, and flasks, which generally may have hydrophilic surfaces to enhance adhesion of cells for growth in culture. It will be understood that the shape or form of a surface that is adapted for cell attachment can vary and may include shapes such as tubes, straws, etc.

In some embodiment, the surface on which the ES cells may attach is a layer of 3T3 cells (e.g. 3T3-J2 cells) or other strain of embryonic fibroblast that are on the surface of a dish or other container. In some embodiments, the support layer of cells, which are also known as “feeder cells” can be cells such as 3T3 cells or other strains of embryonic fibroblasts, that are mitotically inactivated embryonic fibroblast cells. In some embodiments, of the invention, mitotically inactivated cells are irradiated cells, examples of which are irradiated 3T3 cells or other strain of embryonic fibroblast. The ES cell aggregate, when placed on the 3T3 cells, or other suitable strain of embryonic fibroblast, will under appropriate conditions give rise to differentiated progeny that will grow and migrate away distally from the aggregate.

The methods of the invention include culturing an ES cell aggregate under conditions that will support the survival of ES cells and the differentiation, growth, and survival of keratinocytes. Thus, examples of conditions for culture of ES cells that are useful in the methods of the invention may include culture of ES cells in the presence of an irradiated 3T3 support cell layer and cell culture medium, such as cFAD medium (Allen-Hoffmann, B. L. & Rheinwald, J. G., Proc. Natl. Acad. Sci. USA 81: 7802-7806, 1984; Simon, M. & Green, H., Cell 40: 677-683, 1985.). Other conditions that permit ES cell survival and keratinocyte growth may include culture with an alternative cell culture media known in the art and also may or may not include the presence of a supporting cell layer, e.g. an irradiated 3T3 cell layer or other strain of embryonic fibroblast.

When ES cell aggregates (e.g. embryoid bodies and/or mega embryoid bodies) are cultured under conditions that permit progeny cells to grow and migrate, these cells will migrate away from the cell aggregate. As used herein, the term “distally” means away from the cell aggregate location. As can be envisioned by one of skill, the migration distally from the cell aggregate may be in any direction in which a surface that will support the growth of the ES cells is available. For example, an ES cell aggregate may be placed in the center of a surface that will support growth of the ES cells and somatic cells formed from the aggregate may migrate in any or all directions from the aggregate. As this occurs there may be a migration front marking the outer boundary of the migrating cells. As used herein, the term “migration front” means the area of migrating cells that is most distant from the aggregate, when cells are growing distally from the aggregate. The migration front of a distally migrating ES cell will be at the peripheral region of the migrating ES cell area. Thus, as used herein, the term “peripheral region” means a region of a cell that is distal from the aggregate that may be at or near the migration front of the migrating ES cells. Nearly all the somatic progeny of the ES cell aggregate will be located between the aggregate and the migration front. Generally speaking, the periphery would embrace the region at least 60% toward the migration front from the aggregate, at least 70%, at least 80%, and preferably at least 90% toward the migration front from the aggregate.

The invention also involves the isolation of somatic cells growing and migrating from the aggregate, which may be located the peripheral region of the cells growing and migrating distally, which can then be further cultured under suitable conditions. Thus, the invention involves in part, culturing an aggregate of human embryonic stem cells on a surface adapted for cell attachment, for a time sufficient to permit somatic cells to grow and migrate distally from the aggregate.

The length of time sufficient to permit cells to grow and migrate distally ranges from 1 day to 10 or more days, depending, in part, on the source of the cells in the culture. For example, in some embodiments, for cells of an aggregate that is a embryoid body, a time sufficient to permit cells to grow and migrate distally is at least about 10 days. Thus, in some embodiments of the invention, the cells are permitted to grow and migrate distally from the aggregate for about 9, 10, 11, 12, 13, 14, 15, 16, 17, 18, 19, 20, 21, 22, 23, 24, or 25 days. Cells of keratinocyte lineage can be isolated from the peripheral region at least about 10 days after the ES cell aggregate culture is initiated when the ES cell aggregate is an embryoid body. The isolated cells may then be cultured under conditions that support the growth of keratinocytes and will form colonies and may undergo stratification. It is important to select cells that are located at or near the migration front at 10 or more days after initiation of the ES aggregate culture, because the distance from the ES aggregate positively correlates with the commitment of a migrating cell to a keratinocyte fate. In some embodiments, the time between initiation of ES cell aggregate culture and the isolation of a cell of keratinocyte lineage from a peripheral region of the cells growing and migrating distally from the aggregate is about 8, 9, 10, 11, 12, 13, 14, 15, 16, 17, 18, 19, 20, 21, 22, 23, 24, 25, 26, 27, 28, 29, 30, or more days. Optionally, the number of days after initiation of the ES cell aggregate culture of cells are permitted to grow and migrate prior to their isolation from the peripheral region, can be determined based on staging results obtained from a control ES cell aggregate culture as described below.

In some embodiments of the invention, the aggregate of human embryonic stem cells cultured is a mega-embryoid body. For an aggregate that is a mega-embryoid body, the time sufficient to permit cells to grow and migrate distally from the mega-EB is at least about 1 day. Thus, in some embodiments of the invention, the cells are permitted to grow and migrate distally from the mega-EB for at least about 1, 2, 3, 4, 5, 6, 7, 8, 9, 10, 11, 12, 13, 14, or 15 days. Cells of keratinocyte lineage can be seen in the zone of migration within as little as 1 day and may be isolated from the peripheral region at least about 1 day after the ES cell aggregate culture is initiated. At a later time, when keratinocytes are formed, they may be isolated and cultured under conditions that support their growth, they will then form colonies may undergo stratification. It is important to select cells thai are located at or near the migration front at 1 or more days after initiation of the ES aggregate culture, because the distance from the ES aggregate positively correlates with the commitment of a migrating cell to a keratinocyte fate. In some embodiments, the time between initiation of ES cell aggregate culture and the isolation of a cell of keratinocyte lineage from a peripheral region of the cells growing and migrating distally from the aggregate is about 1, 2, 3, 4, 5, 6, 7, 8, 9, 10, 11, 12, 13, 14, 15, 16, 17, 18, 19, 20, 21, 22, 23,24, 25,26, 27,28,29, 30, or more days. Optionally, the number of days after initiation of the ES cell aggregate culture that of cells are permitted to grow and migrate prior to isolating cells from the peripheral region, can be determined based on staging results obtained from a control ES cell aggregate culture as described below.

Following the isolation of a cell from the ES aggregate culture, the cell may be cultured under conditions to permit colonization and/or stratification of keratinocytes. In some embodiments, of the invention, conditions that permit colonization of keratinocytes, are culture of the isolated cells on irradiated 3T3 cells in the presence of cFAD. Alternative conditions, which are known in the art to permit colonization and/or stratification of keratinocytes may also be used in the methods of the invention. An example of an alternative condition, though not intended to be limiting, is culture of a cell of keratinocyte lineage isolated from the ES aggregate culture in serum-free medium. Alternative keratinocyte culture conditions that are useful in methods of the invention may or may not include the use of a support cell layer, such as an irradiated 3T3 support cell layer or support layer of other strain of embryonic fibroblast. Other conditions are suitable to permit colonization of keratinocytes, and these alternatives will be known to those of skill in the art. In some embodiments, keratinocytes will also be cultured to permit stratification, and these methods are also known to those of skill in the art.

Additional conditions that are useful to permit colonization of keratinocytes include first culturing ES cell-derived keratinocytes for one or more passages in low-Ca⁺⁺ serum-free medium with or without irradiated 3T3 cells or other strain of embryonic fibroblast and subsequently culturing the ES cell-derived keratinocytes for one or more passages in cFAD medium (or other suitable medium) with or without irradiated 3T3 cells or other strain of embryonic fibroblast. In some embodiments of the invention, the cFAD medium also includes PCS, for example 10% (v/v) FCS.

When the culture of the isolated keratinocytes is established, the keratinocytes can be expanded in culture to form sheets or cultured using any art-known strategies. As used herein the term “expanded” means grown with an increase in cell number. Thus, to expand a cell in culture is to have that cell divide and have more cells produced from that cell and its progeny, forming a colony of ES cell-derived keratinocytes. Thus, expansion of a keratinocyte cell in culture will result in an increase in the number of keratinocytes in culture and can result in the formation of cell colonies.

Keratinoctyes obtained through the methods of the invention can be used in methods of treatment used in various ways. One example, though not intended to be limiting, is expanding in culture a keratinocyte isolated with the methods of the invention to make a keratinocyte sheet. Thus, the keratinocyte cells in culture expand in number and generate a keratinocyte sheet. A sheet of keratinocytes is a confluent area of keratinocytes. Sheets may be formed by fusion of keratinocyte cell colonies in culture. Under culture conditions such as those described herein and others known in the art, colonies of keratinocytes can grow, fuse and form single or multilayered confluent keratinocyte sheets. Keratinocyte sheets generated with the method of the invention can be used in research compositions and methods as well as in therapeutic methods to treat conditions such as the loss of skin through wounds, burns, disease, or other trauma.

In addition to an ES cell aggregate culture from which cells of keratinocyte lineage can be isolated, the methods of the invention also encompass the preparation of a control culture of an ES cell aggregate. As used herein, the term “control culture” means a culture that is prepared and cultured in parallel (e.g. under identical conditions) with an ES cell aggregate culture from which a cell of keratinocyte lineage will be isolated. Although not required to practice the methods of the invention, a control culture can be useful in that it allows the histological-based staging of the ES cells to determine the stage of progression of the cells from ES cells to keratinocytes. Histological-based staging can be done on the control culture cells for example using any test to identify markers present in the cells. For example, antibody-based labeling, of cells growing and migrating distally from an ES aggregate can be performed to indicate the presence of absence of marker proteins in the cultured cells. The presence or absence of transcription markers such as p63 and basonuclin and differentiation markers such as K14 and involucrin can be determined using methods known in the art, including the antibody-based methods described in the Examples section. As described herein, the determination of the status of the markers correlates with development of the keratinocytes lineage toward the stage at which differentiated keratinocytes can be isolated. Thus, a control culture may serve and assist in the staging of the ES cell aggregate culture from which cells of keratinocyte lineage will be isolated.

The invention also relates, in part, to determining circumferential movement in ES cell-derived keratinocytes. Circumferential movement is present in ES cell-derived keratinocytes in the presence of retinoic acid. Circumferential movement is not observed in fetal or post-natal keratinocytes, or other ES, fetal, or post-natal cell types. Circumferential movement can be used as a marker for cells that can be harvested and expanded for use in research methods and in therapeutic treatments. Cells that show circumferential movement have protein expression that differs from cells that do not show circumferential movement. In some aspects of the invention, proteins that are specifically expressed in cells with circumferential movement can be used as markers for cells (e.g. keratinocytes) that can be expanded for use in therapeutic treatment methods for skin wounds, burns, disease, or other trauma

As used herein, the term “circumferential movement” means movement of the cell membrane and sub-adjacent cytoplasm in a circular direction at sufficient rapidity to be observed with real-time imaging. Thus, the cell membrane and sub-adjacent cytoplasm move around in a circular manner along the circumference of the cell. In some embodiments, the concentration of retinoic acid with which the cells are contacted is at least about from about 10⁻⁷, 10⁻⁸, 10⁻⁹ through 10⁻¹⁰ molar retinoic acid in the culture medium. In the presence of retinoic acid, ES-derived keratinocyte cells engage in a form of circumferential movement of the cell membrane and sub-adjacent cytoplasm.

The circumferential movement in cells can be determined visually under the microscope and can be recorded by imaging methods known in the art, including, but not limited to photography, video imaging, etc. Methods to determine the presence of circumferential movement include, but are not limited to microscopy. An example of a microscopy method useful in the methods of the invention is phase microscopy. The methods of the invention also include determining the presence of circumferential movement in ES cell-derived keratinocytes, and comparing that determination to the determination of circumferential movement in control cells such as other cells types, fetal keratinocytes, and/or post-natal keratinocytes.

The determination of circumferential movement in an ES cell-derived cell is useful for identifying an ES cell-derived cell that can be used in therapeutic methods for treating skin injury or disease in a subject. Identifying cells with circumferential movement, allows the selection of such cells for treatment of skin wounds, burns, disease, or other trauma. Methods of the invention relating to cell selection include contacting an ES cell-derived cell with retinoic acid, determining the presence of circumferential movement in the contacted cell, and if circumferential movement is present, the cell is identified as a cell that is useful for treating injury in the subject. In some embodiments, the ES cell-derived cell is an ES cell-derived keratinocyte. After identification of a cell with circumferential movement, the cell can be expanded in culture (e.g. grown into sheets) as described herein or using any art-known method, for use in research compositions and/or methods as well as in therapeutic methods to treat conditions, such as the loss of skin through burns or trauma.

The invention also relates, in part, to determining fragmentation of colonies of ES cell-derived keratinocytes. Fragmentation of colonies is not observed in fetal or post-natal keratinocytes, or other ES, fetal, or post-natal cell types. Fragmentation of colonies can be used as a marker for cells that can be harvested and expanded for use in therapeutic treatments. Keratinocyte colonies that show fragmentation have keratinocyte cells in which protein expression that differs from cells that do not show colony fragmentation. In some aspects of the invention, proteins that are specifically expressed in cells with colony fragmentation can be used as markers for cells (e.g. keratinocytes) that can be expanded for use in research compositions and methods as well as in therapeutic treatment methods for skin wounds, burns, disease, or other trauma As used herein, the term “colony fragmentation” means that colonies that are formed by ES cell-derived keratinocytes in culture break apart and form smaller colonies.

The methods of the invention also relate, in part, to treatment of skin wounds, burns, disease, or other trauma. Skin damage may be the result of disease or injury and include any condition that can be treated by the administration of the ES cell-derived keratinocytes of the invention. Skin damage may also include skin erosion and the effects of aging. Burn injuries that can be treated using the ES cell-derived keratinocytes cells and the methods of the invention include, but are not limited to: heat, chemical, UV, and electrical burns.

The ES cell-derived keratinocytes of the invention and ES cell-derived keratinocytes derived using the methods of the invention, can be used to treat skin trauma or injury. In some embodiments, sheets of ES cell-derived keratinocytes can be administered to a subject in need of such treatment. In other embodiments, individual ES cell-derived keratinocytes or groups of ES cell-derived keratinocytes may be administered to a subject in need of such treatments. The application of cells and/or sheets of cells to a subject for wound treatment is well known in the art. Art-known methods for used for keratinocyte expansion, transfer and administration can be used in conjunction with the ES cell-derived keratinocytes described herein. It will be understood that the ES cell-derived keratinocytes of the invention can be used alone or can be combined with additional cell types, materials. or solutions for administration to a subject for treatment of skin wound or trauma. For example, in some embodiments, ES cell-derived keratinocytes of the invention may be combined with a mesh or other support material for administration to a subject. Those of skill in the art will understand that additional art-known methods of administering keratinocytes or sheets of keratinocytes for therapeutic methods can be used in conjunction with the methods and products of the invention.

The invention will be more fully understood by reference to the following examples. These examples, however, are merely intended to illustrate the embodiments of the invention and are not to be construed to limit the scope of the invention.

### EXAMPLES

### Example 1

Introduction

Human embryonic stem cells injected into scid mice produce nodules containing differentiated somatic tissues. From the trypsinized cells of such a nodule, we have recovered keratinocytes that can be grown in cell culture. The method of recovery is sensitive enough to detect small numbers of keratinocytes formed in the nodule, but for purposes of analysis, it is preferable to study the development of the entire keratinocyte lineage in culture. The principle of our analysis is the successive appearance of markers, including transcription factors with considerable specificity for the keratinocyte (p63 and basonuclin) and differentiation markers characteristic of its final state (keratin 14 and involucrin). We have determined the order of marker succession during the time- and migration-dependent development of keratinocytes from single embryoid bodies in cell culture. Of the markers we have examined, p63 was the earliest to appear in the keratinocyte lineage. The successive accumulation of later markers provides increasing certainty of emergence of the definitive keratinocyte.

Methods

The human ES cell line H9, which is used in all these experiments, was derived at the University of Wisconsin by D. A. Thomson and coworkers (Thomson, J. A. et al., Science 282: 1145-1147, 1998.). For preparation of fibroblast feeders, fibroblasts of 13-day mouse embryos (PMEF-H) treated with Mitomycin C were purchased from Specialty Media (Phillipsburg, N.J.), and 3T3-J2 cells were as reported in Rheinwald, J. G. & Green, H., Cell 6: 331-343, 1975 and Allen-Hoffmann, B. L. & Rheinwald, J. G., Proc. Natl. Acad. Sci. USA 81: 7802-7806, 1984. Marker proteins detected by specific antibodies were as follows: Oct4 (Santa Cruz Biotechnology, Inc. Santa Cruz, Calif.), p63 [with the 4A4 monoclonal antibody (Yang, A. et al., Mol. Cell 2: 305-316, 1998.), provided by F. McKeon and A. Yang], basonuclin (Iuchi, S. & Green, H., 1997 Proc. Natl. Acad. Sci. USA 94: 7948-7953.), involucrin (Biomedical Technologies, Stoughton, Mass.), and K14 (Chemicon International, Temecula, Calif.). Staining of Western blots of keratinocyte extract with the K14 antiserum revealed a single strong band corresponding to the expected molecular weight of 50,000. Extracts of cultured H9 cells submitted to Western blot analysis and staining with a pan keratin antibody revealed no labeled bands with a molecular weight ˜81,000.

Culture medium for growing ES cells was as described (Schuldiner, M. et al., Proc. Natl. Acad. Sci. USA 97: 11307-11312,2000.), without lymphocyte-inhibitory factor. For experiments on detection and isolation of keratinocytes, including experiments on attached embryoid bodies, we used cFAD medium (Allen-Hoffmann, B. L. & Rheinwald, J. G., Proc. Natl. Acad. Sci. USA 81: 7802-7806, 1984; Simon, M. & Green, H., Cell 40: 677-683, 1985.) with or without subsequently added irradiated 3T3-J2 supporting cells.

Transcription Factors Used as Markers

p63 is a transcription factor whose gene and transcripts were first fully described by F. McKeon and coworkers and whose expression they showed to be specific for keratinocytes and related epithelial cell types (Yang, A. et al., Mol. Cell 2: 305-316, 1998.). Disruption of the gene results in failure of development of the epidermis, all other stratified squamous epithelia, and a few related epithelia such as mammary, sebaceous and lacrimal gland, prostatic, urothelial, and cervical (Yang, A. et al., Mol. Cell 2: 305-316, 1998; Yang, A. et al., Nature 398: 714-718, 1999; Mills, A. A. et al., Nature 398: 708-713, 1999; O'Connell, J. T. et al., Gynecol. Oncol. 80: 30-36, 2001; Signoretti, S. et al., Am. J. Pathol. 157: 1769-1775, 2000.). p63 is thought to be necessary for the maintenance of stem cell precursors (Yang, A. et al., Nature 398: 714-718, 1999; Pellegrini, G. et al., Proc. Natl. Acad. Sci. USA 98: 3156-3161, 2001.) and is present in all growing cells of keratinocyte colonies with high growth potential (holoclones, Barrandon, Y. & Green, H., Proc. Natl. Acad Sci. USA 84: 2302-2306, 1987). In the human, even heterozygous mutations in p63 produce developmental defects of ectodermal structures (van Bokhoven, H. & McKeon, F., Trends Mol. Med. 8: 133-139, 2002; Celli, J. et al., Cell 99: 143-153, 1999; McGrath, J. A. et al., Hum. Mol. Genet. 10: 221-229, 2001; van Bokhoven, H. et al., Am. J. Hum. Genet. 69: 481-492, 2001.).

Basonuclin is a transcription factor containing three separated pairs of zinc fingers (Tseng, H. & Green, H., Proc. Natl. Acad. Sci. USA 89: 10311-10315, 1992; Iuchi, S. Cell. Mol. Life Sci. 58: 625-635, 2001.). It is present in basal cells of the epidermis and other squamous epithelia (Tseng, H. & Green, H., J. Cell Biol. 126: 495-506, 1994.). In rapidly growing cultured keratinocytes (Tseng, H. & Green, H., J. Cell Biol. 126: 495-506, 1994; Iuchi, S. et al., Exp. Dermatol. 9: 178-184, 2000.) and in squamous tumors (Parsa, R. et al., J. Invest. Dermatol. 113: 1099-1105, 1999.), it is concentrated in cell nuclei, but in the normal epidermis or in keratinocytes cultured under conditions not optimal for cell growth, it may be cytoplasmic (Iuchi, S. et al., Exp. Dermatol. 9: 178-184, 2000.). Nuclear localization of basonuclin may result from the absence of phosphorylation of Ser-541, located immediately C-terminal of the nuclear localization signal (Iuchi, S. & Green, H., 1997 Proc. Natl. Acad. Sci. USA 94: 7948-7953.).

Differentiation Markers

K14 is a keratin of the basal cells of all stratified squamous epithelia (Moll, R. et al., Cell 31: 11-24, 1982; Quinlan, R. A. et al., Ann. N.Y. Acad. Sci. 455: 282-306, 1985; Galvin, S. et al., Adv. Dermatol. 4: 277-300, 1989.). Involucrin is a protein precursor of the cross-linked envelope that forms late in the terminal differentiation of the keratinocyte (Rice, R. H. & Green, H. Cell 18: 681694, 1979.). In contrast to K14, involucrin is made only in suprabasal cells (Banks-Schlegel, S. & Green, H. J. Cell Biol. 90: 732-737, 1981; Watt, F. M. & Green, H., Nature 295: 434-436, 1982.).

Localization of p63, Basonuclin, and K14 in Keratinocyte Colonies.

A keratinocyte colony was formed with 3T3 support from cells of an ES cell-produced nodule in a scid mouse. A large nodule resulting from ES cells injected into the leg muscle of a scid mouse was excised, minced, and trypsin-disaggregated. Cells (10³) from the second trypsinization were plated on 3T3 feeders and fed with cFAD medium. Eleven days later, a colony with morphology typical of keratinocytes was seen under phase microscopy. The colony was fixed and stained for p63, basonuclin, and K14. Nuclear p63, basonuclin, and cytoplasmic K14 were evident in the cells. In separate images for each staining, the two transcription factors were detectable in almost all cells, although not with identical intensity. K14 was present everywhere but was particularly marked at the expanding perimeter of the colony.

Assay for Involucrin in a Keratinocyte Colony

The presence of involucrin was assayed in a keratinocyte colony derived as described above herein. A primary colony formed with 3T3 support was fixed and stained on day 20. Each cell containing p63 also contained K14. Most regions of the colony contained involucrin. In squamous epithelium, K14 synthesized in the basal layer persisted in the suprabasal layers (Roop, D. R. et al., Cancer Res. 48: 3245-3252, 1988.) but not in cornified cells. Our results indicated that the cells brightly stained for K14 did not contain appreciable involucrin, whereas cells containing involucrin were faintly stained for K14. It appeared that complete destruction of K14 had not yet taken place.

Disappearance of Oct4 from Cells Migrating Out of an Embryoid Body.

A single embryoid body was deposited on a tissue culture dish. Five days later, the culture was fixed and stained for Oct4. The direction of migration was away from the embryoid body. The cells of the embryoid body stained brightly for nuclear Oct4. A few cells located in the migration zone close to the embryoid body retained detectable Oct4 but beyond this, very few of the 4˜,6-diamidino-2-phenylindole stained nuclei contained even a trace of Oct4.

Appearance of p63and K14 in Migrating Cells

We examined the appearance of p63 and K14 in cells migrating from an attached embryoid body inoculated 15 days previously. Of 517 p63-containing cells, only 30 also contained K14. Because K14 is a cytoplasmic protein, it extends beyond the corresponding p63-containing nucleus. No K14-containing cell lacked p63. Cells lacking both markers were revealed by 4˜,6-diamidino-2-phenylindole staining for DNA. No supporting feeders were present, indicating that cells lacking p63, although they lacked distinctive morphology, belonged to non-keratinocyte human lineages.

Cells of Keratinocyte Lineage Appear Close to the Migration Front

We examined the location of cells of the keratinocyte lineage after migration using K14, p63, and basonuclin staining. After migration from an embryoid body for 27 days, the migration front showed numerous cells with K14, p63, and basonuclin staining. At higher power microscopic examination, the presence of cytoplasmic K14 could be correlated with the presence of nuclear basonuclin staining. Other cells containing only p63 were at an earlier stage in the development of the keratinocyte lineage.

Appearance of Involucrin in a Keratinocyte

We examined the appearance of involucrin in a stratified keratinocyte colony originating from the migration region of a cultured embryoid body. After 13 days of migration from an embryoid body, the cells were trypsinized and inoculated onto 3T3 feeders. Twenty-four days later, a culture containing a colony with the appearance of keratinocytes was fixed and stained. The results indicated that nearly all cells contained p63 and most cells appeared to contain K14. Scattered squamous-like regions overlying the basal layer contained involucrin.

Keratinocyte Lineage Appears at the Migration Front

We analyzed the concentration of the keratinocyte lineage at the migration front after the addition of 3T3 cells. After allowing migration from an attached embryoid body for 8 days, 2.6˜10⁴ irradiated 3T3 cells per cm² were added to the culture, and incubation was continued for 19 days. At that time, the zone close to the migration front was nearly completely composed of cells containing p63, K14, and basonuclin.

Results

Demonstration that the Culture System Supports the Multiplication of Keratinocytes Generated from Human ES Cells Injected into scid Mice.

Two months after injection of 10⁷ H9 cells into scid mice, the resulting nodules were trypsin-disaggregated, and the cells were inoculated into dishes containing supporting irradiated 3T3 cells and cFAD medium. Under phase microscopy, colonies with the morphology of keratinocytes were obtained. Staining the same colony for p63, basonuclin, and K14 showed that all three markers were present.

One such colony was allowed to grow for 20 days. This time allowed stratification and terminal differentiation. Immunostaining of such a colony for involucrin showed that most regions of the colony contained K14 and involucrin, but some regions contained only K14, suggesting the absence of stratification in those regions. These experiments demonstrated that the colonies whose founding cell originated from nodules formed in scid mice were keratinocytes, although they may not be identical with keratinocytes cultured from epidermis of postnatal humans.

From serial photographs of several different living colonies and cell counts carried out on enlarged prints, we obtained cell-doubling times of 16-22 h, up to a colony size of 1,000 cells. We made secondary and tertiary subcultures of such colonies, but in these experiments we did not obtain holoclones with persistent high growth potential.

Using this detection system, we recovered keratinocyte colonies at a frequency of about 1 per 10⁴ cells plated. This sensitivity is evidently greater than that afforded by histological sections stained with hematoxylin˜eosin, because in serial sections of the nodules we were unable to positively identify any stratified squamous epithelium among the differentiated tissues formed.

Differentiation from Cultured ES Cells: The First Step Marked by the Loss of Oct4.

ES cells are known to contain the germ-line-specific nuclear transcription factor Oct4, a member of the POU family (Scholer, H. R. et al., EMBO J. 9: 2185-2195, 1990; Palmieri, S. L. et al., Dev. Biol. 166: 259-267, 1994; Scholer, H. R. et al., EMBO J. 8: 2543-2550, 1989; Rosner, M. H. et al., Nature 345: 686-692, 1990; Scholer, H. R. Trends Genet. 7: 323-329, 1991, Scholer, H. R. et al., EMBO J. 8: 2551-2557, 1989.). To study the early development of the keratinocyte lineage beginning with cells containing Oct4, we deposited, in the middle of a 60-mm tissue culture dish, a single embryoid body previously prepared from aggregated ES cells. The embryoid body quickly attached to the surface, and centrifugal cell migration began from its perimeter. Five days later, the embryoid body still contained Oct4. In the zone of migration, Oct4 began to disappear quite close to the embryoid body and was absent from nearly all cells located close to the migration front. We then examined how the migrating cells developed along a keratinocyte lineage.

p63: An Early Marker of the Keratinocyte Lineage.

In 5-day cultures of an embryoid body, we detected in the nearby migrating region, a few cells with nuclei containing p63. At 15 days we found large clusters of such cells, located at some distance from the embryoid body. Only 5.8% of the p63-containing cells also possessed K14. No K14 could be identified in cells not containing p63.

The Order of Appearance of Basonuclin and K14 in p63-Containing Cells.

After 27 days of migration, p63-containing cells were abundant close to the migration front. Of 114 such cells, 50 (or 44%) also contained K14. These cells often contained basonuclin as well.

In the part of the migration zone located close to the embryoid body (far behind the front), cells containing p63, even when numerous, did not contain basonuclin. In this region, we observed a few cells that definitely contained basonuclin, but no p63. These might be primordial germ cells, which are known to contain basonuclin (Mahoney, M. G. et al., Biol. Reprod. 59: 388-394, 1998; Tian, Q. et al., Development (Cambridge, U.K.) 128: 407-416, 2001; Yang, Z. et al., Proc. Natl. Acad. Sci. USA 100: 11457-11462, 2003.) and which have recently been shown to develop in cultured embryoid bodies (Toyooka, Y. et al., Proc. Natl. Acad. Sci. USA 100: 11457-11462, 2003.). It was only in cells that had advanced further from the embryoid body that basonuclin appeared in cells of the keratinocyte lineage, because these cells also contained p63. The relation between the times of appearance of basonuclin and K14 in the keratinocyte lineage was examined in the middle region of the migration zone by scoring those cells containing p63 and K14 but no basonuclin and those cells containing p63 and basonuclin but no K14. In four experiments, we found 167 cells in the first category and 26 in the second. This finding indicates that, in 86% of the total, the appearance of K14 preceded that of basonuclin. We conclude that after the appearance of p63, K14, and basonuclin appear nearly simultaneously; in general, K14 is detected earlier.

Formation of Involucrin by Cells Migrating from an Embryoid Body.

After a 13-day period of cell migration from an embryoid body, the cells were trypsinized and inoculated onto a culture containing a feeder layer of 3T3 cells and fed with cFAD medium. Some slowly growing keratinocyte colonies developed. Like keratinocytes cultured from the nodules formed in scid mice, these colonies were found to contain cells possessing involucrin by immunostaining.

Effect of Addition of 3T3 Cells to a Culture Containing Cells Migrating from an Embryoid Body.

In such experiments, cell migration from an embryoid body was allowed to proceed for 8 days and then lethally irradiated 3T3 cells were added to a density of 2.6˜10⁴ per cm². After an additional 19 days, nearly all the cells close to the migration front contained p63, K14, and basonuclin. These conditions therefore resulted in concentration, close to the migration front, of an almost pure population containing the three important markers of the keratinocyte.

Discussion

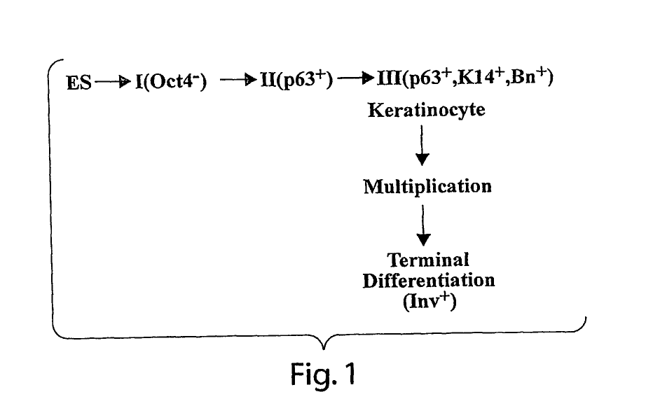

The results of the above-described studies are summarized in FIG. 1. As illustrated in FIG. 1, stages I, II, and III are consecutive. Stage I is defined by the disappearance of Oct4. An interval of time and a degree of cell migration follow before the first marker of the keratinocyte lineage, p63, appears. The presence of p63 in the absence K14 and basonuclin defines stage II. Such cells are quite numerous early in the process of cell migration. This finding seems consistent with what is known about then appearance of p63 in mouse embryogenesis, for recent studies have found p63 as early as stage E7.5, whereas K5 (partner of K14) does not appear until E12 (F. McKeon, personal communication). It may be postulated that, in human embryogenesis (a slower process than in the mouse), p63-containing cells lacking the later markers should be more numerous.

With further time and migration, K14 and basonuclin appear (stage III). It appears that basonuclin, which is present in the male and female germ line (Yang, A. et al., Mol. Cell 2: 305-316, 1998; Mahoney, M. G. et al., Biol. Reprod. 59: 388-394, 1998; Tian, Q. et al., Development (Cambridge, UK.) 128: 407-416, 2001.), must disappear soon after fertilization, because we did not detect it in ES cells, and then reappear late in the development of the keratinocyte lineage. Because basonuclin and K14 appear at nearly the same time and K14 is not always the first to be detected, we simply represent the acquisition of both markers as necessary for the transition to stage III. Once p63 has appeared, the other markers of the keratinocyte follow progressively with time and cell migration from the embryoid body. The proportion of p63-containing cells bearing later markers increased from 5.8% to 44% to nearly 100%.

The keratinocytes identified in our experiments have not been assigned to a particular squamous epithelium (epidermal, oral, esophageal, etc.). This identification is done by examining differentiation markers of the suprabasal layer where that layer is well developed, as in epithelia grafted to animals. In rodent keratinocytes, such identification may be complicated by metaplastic changes (Phillips, M. A. & Rice, R. H. J. Cell Biol. 97: 686-691, 1983; Parenteau, N. L. et al., Differentiation (Berlin) 33:130-141, 1986.), but this complication is less likely in human keratinocytes.

The entire developmental lineage of the keratinocyte can, in principle, be defined by immunostaining for transcription factors known to be components of the keratinocyte (Eckert, R. L. et al., J. Invest. Dermatol 109: 501-509, 1997) or for transcription factors (and their coactivators) that are not yet known in keratinocytes or that might be confined to their precursors. Once the pattern of marker succession has been established, the importance of any given transcription factor can be determined by MRNA ablation by using any of several methods now available.

### Example 2

Background

Much of the general enthusiasm for research on human embryonic stem cells is based on the possible therapeutic use of derived somatic cell types. Several issues arise in this connection: Because ES cells are capable of forming teratomas, it is important to free these cell types from all remaining ES cells before their use. To achieve purity, it is important that the derived somatic cell type of interest be made serially cultivable, so it can be clonally isolated. There are currently no examples cited in the literature for any cell type derived from human ES cells.

In addition, the somatic cell types derived from ES cells must be examined to determine whether they are identical to the similar somatic type isolated from post-natal or fetal tissues. ES cells are a cultured cell type, not an implanted blastocyst; therefore, clues that establish order in embryos (such as polarity and gradients) are absent. If important cues are missing, the cell types generated are not necessarily identical to those of fetal and post-natal tissues even though they may possess most or all of the known markers of keratinocytes.

We have examined these issues that apply to the development and use of embryonic stem cells.

Culture Conditions for Support of Multiplication of Keratinocytes Derived from ES Cells

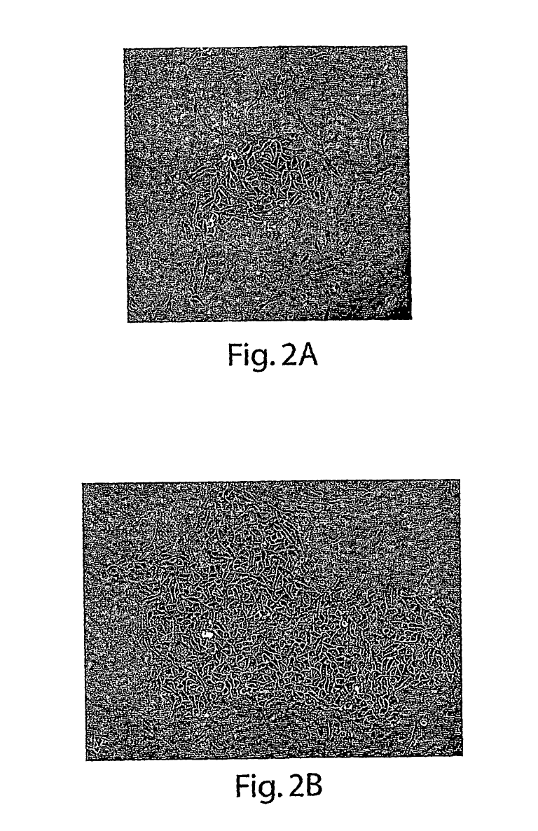

It is important to consider that keratinocytes arising in scid mice from ES cells when transferred to culture grow very well for a short period (FIG. 2 shows such a colony in rapid growth). But on serial transfer, the growth potential of such colonies deteriorates. This seems to mean that the culture systems that have previously developed for growing post-natally derived keratinocytes are not optimal for the continuing growth of ES-cell derived keratinocytes. This problem is not only a practical one but also must have biological meaning for the development of the keratinocyte lineage.

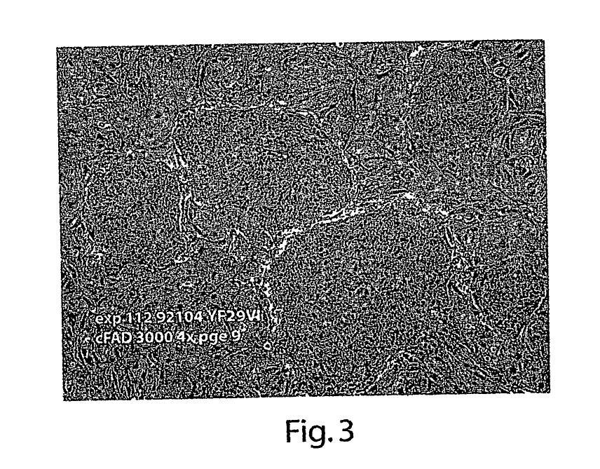

The 3T3 support system developed in my laboratory many years ago produces the quality of multiplication of fetal or post-natal keratinocytes shown in FIG. 3. The keratinocyte colonies expand by excavating the adjacent irradiated 3T3 cells and produce colonies with a frequency of 20-60% of cells plated. Many of these colonies have smooth round borders and are likely to be holoclones. A smaller number are wrinkled colonies which have aborted or will abort. Cultures with an adequate number of holoclones can be grown through 150 cell generations in culture.

We have now determined that the use of supporting 3T3 cells for the cultivation of keratinocytes derived from ES cells allows us to obtain about 20 cell generations in culture. This is an improvement over the past generational numbers obtainable. We have isolated clones and are working to expand them to mass cultures. In one set of experiments, frozen cells of ES cell nodule H9-3 T3 in scid mice and never grown in culture were thawed and inoculated into culture containing irradiated 3T3 cells and Cascade (EpiLife) medium (Cascade Biologics, Portland, Oreg.—passage I). Ten days later, the cells were transferred to irradiated 3T3 cells+Cascade (Passage II). Ten days later the cells were again transferred to irradiated 3T3 cells+Cascade (Passage III). After approximately five weeks, the cells were transferred to cFAD medium containing 10% (v/v) fetal calf serum with irradiated 3T3 cells and transferred regularly under same conditions thereafter. The cells were grown through 20.6 generations using this method. Details of cell generation from the experiment are shown in Table 1.

TABLE 1
Cell Generations with two-stage culture method.

| Passage | Dilution | Generations | Total Generations |
|---|---|---|---|
| End of II | 1:40 | 5.2 | 5.2 |
| End of III | 1:4 | 2 | 7.2 |
| End of IV | 1:4 | 2 | 9.2 |
| End of V | 1:25 | 4.5 | 13.7 |
| End of VI | 1:5 | 2.3 | 16.0 |
| End of VII | 1:5 | 2.3 | 18.3 |
| End of VIII | 1:5 | 2.3 | 20.6 |

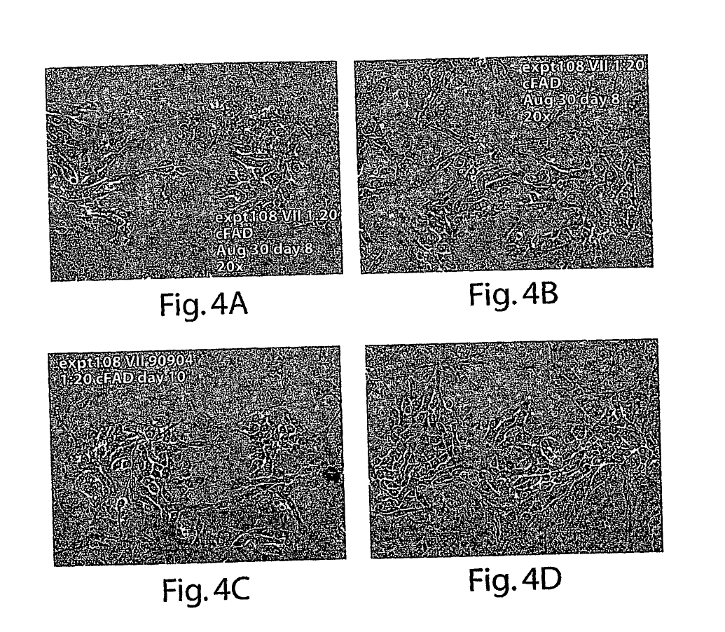

The colonies of ES cell-derived keratinocytes cultivated under the same conditions do not resemble the colonies produced by post-natal keratinocytes cultivated under the same conditions, which are shown in FIG. 3. The ES cell-derived keratinocytes have difficulty in excavating the 3T3 cells, they grow more slowly and they are deformed. They have a very peculiar tendency to undergo fragmentation. FIG. 4 shows four examples in which keratinocytes derived from nodules in scid mice were serially transferred with 3T3 support. FIGS. 4A-D show colonies 8-10 days after plating of passage VII. These colonies are much small than those of post-natal keratinocytes at the same time after inoculation. The colonies are irregular in outline and appear to be breaking up by movement of parts of the colony in opposite directions. The result of colony break-up is that the size of any colony is not a measure of the growth that has occurred since plating. After colony break-up, the number of colonies found will be greater than the starting number of colony-forming cells, and the size of the fragmented colonies will give an underestimate of the growth of the original colony-forming cell.

An important addition we have made to our experiments to improve multiplication is the use of the serum-free low Ca⁺⁺ medium. This medium has the important advantage of eliminating ES cells that otherwise will grow on 3T3 cells and inhibit the growth of any keratinocytes. We have also found that this medium has the serious limitation for the growth of ES cell-derived keratinocytes that the cells will not tolerate dilution in it. We have developed a method of using a two-stage process in which the low Ca⁺⁺ serum-free medium is used to get rid of ES cells and the keratinocytes are then transferred to serum-containing medium with 3T3 support. Although 3T3 cells aren't well maintained over time in the low Ca⁺⁺ medium, we have been able to combine 3T3 support with the low Ca⁺⁺ medium and have obtained five to ten fold higher recovery of keratinocytes from ES-produced nodules.

### Example 3

Mega Embryoid Bodies (EBs) and their Conversion to Keratinocytes

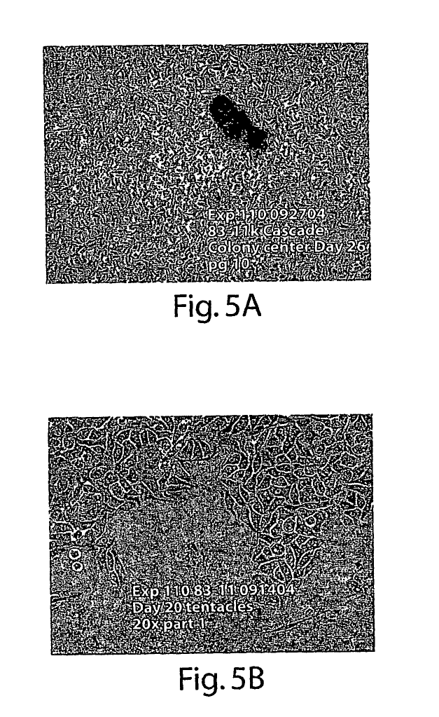

We have developed a new method of preparing EBs so that they are much larger than those prepared by conventional methods. We call them Mega EBs or multilocular EBs. They are produced in a flask whose geometry favors larger scale aggregation on the bottom. We have developed conditions permitting EBs so produced, when transferred to tissue culture dishes, to attach very quickly and cell migration onto the surface of the dish begins almost immediately. Differentiation along the keratinocyte lineage occurs much more rapidly than in earlier experiments. On the very day following introduction of an EB to the tissue culture dish, p63-containing cells appear in the zone of migration and a great many p63-containing cells appear by five days. This suggests that keratinocyte formation from cultured EBs is greatly accelerated in comparison with normal human embryogenesis. In the mouse, p63 appears at between ⅓ and ½ of the course of gestation. A corresponding figure in the human, would be 13-20 weeks of gestation. FIG. 5 shows a colony forming from a rather small embryoid body by migration and multiplication. It appears to consist almost entirely of keratinocytes. The method for the preparation of mega EBs and their attachment to dishes is as follows.

Preparation of EBs

About 2.6×10⁵ H9 ES cells were inoculated into 1×75 cm² flask on 10×10⁶ primary mouse embryonic fibroblasts (PMEF) in SR medium. (Schuldiner, M., et al., 2000 PNAS USA 97(21): 11307-12). The 30 ml of medium was changed every 2 days. The following steps were followed for the preparation of EBs. At Day 5 after inoculation, the colonies about half confluent. The cells were trypsinized and about 21×10⁶ cells were recovered. Approximately 3×10⁶ cells were placed in 4 ml of SR in a Teflon flask and the flask was turned upside down. After 1 hour 40 minutes after placing cells into flask, nice aggregates were present. Three hours after inversion, the flask was turned back to an upright position. Twenty-four hours after the cells were initially placed in the flask, the flask contained nice EBs, some as large as 1 mm in diameter. Some of the EBs were cigar-shaped multi-locular EBs as long as 3 mm. Additional SR medium was added as necessary to prevent acidity

Attachment (Using Day 2 or later EBs)

cFAD medium was placed in a 6 cm dish and distributed uniformly. A single EB was placed in middle of each dish. Twenty-four hours later the EBs had attached and migration had begun; cells with p63 were already detected in migration region using 2 day old EBs. Three ml cFAD (prepared without transferrin) was added to the culture. (Simon, M. and H. Green 1985 Cell 40(3): 677-83).

### Example 4

Analysis of properties of ES Cell-Derived Keratinocytes

As described above, we have identified the following peculiarities in the ES-derived keratinocytes as compared to fetal or post-natal keratinocytes. The ES-derived keratinocytes exhibit a slow growth rate in the presence of 3T3 support (doubling time ˜48 hours compared with less than 24 hours for post-natal keratinocytes). ES-derived keratinocytes have a reduced ability to excavate adjacent 3T3 cells. ES-derived keratinocyte colonies exhibit colony break-up into two or more fragments migrating in different directions. The ES-derived keratinocytes have an intolerance of dilution in the low Ca⁺⁺ medium.

In addition, we have discovered that the ES-derived keratinocytes have an extraordinary form of cell movement that is not seen in fetal or post-natal keratinocytes. In the presence of retinoic acid at 10⁻⁷ molar, the ES-derived keratinocyte cells engaged in a form of circumferential movement of the cell membrane and subjacent cytoplasm. We have examined this phenomenon and have videotaped this movement in real time. The circumferential movement is readily observed when the culture dish is removed from the incubator and examined (while still warm) under phase microscopy. The circumferential movement appears to be novel and unreported prior to our finding.

We are examining whether the ES cell-derived keratinocytes are different in behavior from fetal and post-natal keratinocytes. We are identifying differences in the two immunocytologically detectable markers between ES cell-derived keratinocytes and fetal and post-natal keratinocytes. For this purpose, we conduct a broad survey of the many proteins found more or less specifically in keratinocytes and for which antibodies are available, comparing post-natal with ES cell-derived keratinocytes. The examination of the protein expression allows the identification of markers that distinguish the two types of keratinocytes.

The subject of keratinocyte migration continues to be of great interest in relation to problems of wound healing (Li et al. 2004 J Invest Dermatol 123(4): 622-33). The extraordinary circumferential movements of ES cell-derived keratinocytes described above might be accompanied by unusual translational movements, and we are examining whether these cells may have a practical use in promoting wound healing.

### Example 5

Identification of ES Cell Derived Cells for Treatment of Injury

The presence of circumferential movement is determined in ES cell-derived cells (e.g. ES cell-derived keratinocytes). The determination is done by contacting the ES cell-derived cells with retinoic acid at a final concentration of 10⁻⁷ molar. The ES cell-derived cells are monitored using phase contrast microscopy to determine the presence of circumferential movement. Cells are observed for movement while still warm from culture incubation. The circumferential movement in the contacted cells is determined visually and/or using other imaging methods such as photography, video imaging, etc. In some experiments, markers such as p63, K14, basonuclin, and/or involucrin are also determined for the cells, using methods described above herein.

Cells that are determined to have circumferential movement in an ES cell-derived cell are harvested and cultured using standard methods and/or methods provided in the Examples s above herein. The identified cells are expanded in culture (e.g. into sheets) as described herein and/or using methods known in the art for use in therapeutic methods to treat conditions, such as the loss of skin through burns or trauma.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

We claim:

1. A method of making a substantially pure culture of ES cell-derived keratinocytes comprising:
expanding a keratinocyte cell harvested from an ES cell nodule to obtain a substantially pure culture of ES cell-derived keratinocytes.

2. The method of claim 1, wherein the harvested keratinocyte cell is expanded in low Ca⁺⁺ medium to selectively deplete ES cells from the culture harvested cells.

3. The method of claim 1, wherein the embryonic stem (ES) cell nodule is a human ES cell nodule.

4. The method of claim 1, wherein the embryonic stem (ES) cell nodule is prepared in a scid mouse.

5. The method of claim 1, wherein means of harvesting the keratinocyte cell from the ES cell nodule comprises disaggregation of the ES cell nodule.

6. The method of claim 5, wherein the disaggregation of the ES cell nodule comprises contacting the ES cell nodule with trypsin.

7. The method of claim 1, wherein the harvested keratinocyte cell is expanded in low Ca⁺⁺ medium with or without 3T3 cells or other strain of embryonic fibroblast.

8. The method of claim 7, wherein the low Ca⁺⁺ medium is serum-free medium.

9. The method of claim 1, wherein the harvested keratinocyte cell is expanded in cFAD medium with or without 3T3 cells or other strain of embryonic fibroblast.

10. The method of claim 9, wherein the cFAD medium comprises 10% (v/v) fetal calf serum.

11. The method of claim 1, wherein the harvested keratinocyte cell is first expanded for one or more passages in low-Ca⁺⁺ medium with or without 3T3 cells or other strain of embryonic fibroblasts to produce a cell culture and subsequently the cell culture is further expanded for one or more passages in cFAD medium with or without 3T3 cells or other strain of embryonic fibroblast.

12. The method of claim 11, wherein the low Ca⁺⁺ medium is serum-free medium.

13. The method of claim 11, wherein the CFAD medium comprises 10% (v/v) fetal calf serum.

14. The method of claim 1, wherein the harvested keratinocyte cell is a cell that displays one or more markers selected from the group consisting of: p63, K14, basonuclin, involucrin, colony fragmentation and circumferential movement.

15. A product formed by the method of claim 1.

16. A method of treating a wound comprising administering keratinocytes from the substantially pure culture of ES cell-derived keratinocytes of claim 1 to a wound.

17. A method of treating a wound comprising administering a composition comprising keratinocytes from the substantially pure culture of ES cell-derived keratinocytes of claim 1 to a wound.

18. A method of making a substantially pure culture of embryonic stem (ES) cell-derived keratinocytes comprising:
expanding selectively a keratinocyte derived from cultured embryonic stem (ES) cells to obtain a substantially pure culture of ES cell-derived keratinocytes.

19. The method of claim 18, wherein the embryonic stem cells are an aggregate.

20. The method of claim 19, wherein the aggregate comprises two or more human embryonic stem cells.

21. The method of claim 19, wherein the aggregate is a human embryoid body or a mega-embryoid body.

22. The method of claim 21, wherein the aggregate is cultured on a surface adapted for cell attachment, for a time sufficient to permit cells to grow and migrate distally from the aggregate.

23. The method of claim 22, wherein the cells that migrate distally away from the cultured aggregate are cells of keratinocyte lineage.

24. The method of claim 22, wherein the surface adapted for cell attachment is a cell culture dish.

25. The method of claim 22, wherein the time sufficient to permit cells to grow and migrate distally from the human embryoid body is at least about 10 days.

26. The method of claim 25, wherein the cells are permitted to grow and migrate distally from the human embryoid body for about 10, 11, 12, 13, 14, 15, 16, 17, 18, 19, 20, 21, 22, 23, 24, or 25 days.

27. The method of claim 22, wherein the time sufficient to permit cells to grow and migrate distally from the mega-EB is at least about 1 day.

28. The method of claim 27, wherein the cells are permitted to grow and migrate distally from the mega-EB for at least about 1, 2, 3, 4, 5, 6, 7, 8, 9, 10, 11, 12, 13, 14, or 15 days.

29. The method of claim 19, wherein the aggregate is cultured in cFAD medium on irradiated 3T3 cells or other strain of embryonic fibroblast.

30. The method of claim 18, wherein the keratinocyte is expanded in serum-free medium with or without irradiated 3T3 cells or other strain of embryonic fibroblast.

31. The method of claim 18, wherein the keratinocyte is first expanded for one or more passages in low-Ca⁺⁺ serum-free medium with or without 3T3 cells or other strain of embryonic fibroblast to produce a cell culture and the cell culture is subsequently expanded for one or more passages in cFAD medium with or without 3T3 cells or other strain of embryonic fibroblast.

32. The method of claim 31, wherein the cFAD medium comprises 10% (v/v) fetal calf serum.

33. The method of claim 18, wherein the keratinocyte is a cell that displays one or more markers selected from the group consisting of: p63, K14, basonuclin, involucrin, colony fragmentation, and circumferential movement.

34. A product formed by the method of claim 18.

35. A method of treating a wound comprising administering keratinocytes from the substantially pure culture of ES cell-derived keratinocytes of claim 18 to a wound.

36. A method of treating a wound comprising administering a composition comprising keratinocytes from the substantially pure culture of ES cell-derived keratinocytes of claim 18 to a wound.

37. A method of treating a skin injury in a subject comprising:
administering to a subject in need of such treatment an amount of the culture of ES cell-derived keratinocytes of claim 1 effective to treat the skin injury.

38. The method of claim 37, wherein the skin injury is the result of disease or trauma.

39. The method of claim 38, wherein the trauma is a burn.

40. A method of identifying an ES cell-derived cell for treating an injury in a subject comprising,
contacting an ES cell-derived cell in culture with retinoic acid,
determining the presence of circumferential movement in the contacted cell, wherein the presence of circumferential movement identifies the cell for treating injury in the subject.

41. The method of claim 40, wherein the retinoic acid is at a concentration in the culture of between about 10⁻⁷ molar and about 10⁻¹⁰ molar.

42. The method of claim 40, wherein the ES cell-derived cell is an ES cell-derived keratinocyte.

43. The method of claim 40, wherein the ES cell-derived cell is an ES cell-derived keratinocyte from a culture made with the method of claim 1.

44. A method of identifying an ES cell-derived keratinocyte for treating an injury in a subject comprising,
culturing an ES cell-derived cell, wherein the cell forms a colony,
determining the presence of fragmentation of the colony, wherein the presence of the fragmentation identifies a cell of the colony as an ES cell-derived keratinocyte for treating injury in the subject.

45. The method of claim 44, wherein the ES cell-derived cell is a cell from an ES cell nodule.

46. The method of claim 44, wherein the ES cell-derived keratinocyte is an ES cell-derived keratinocyte from a culture made with the method of claim 1.

47. A composition comprising a embryonic stem cell-derived keratinocyte from a culture made with the method of claim 1.

48. A method of treating a skin injury in a subject comprising:
administering an amount of the culture of ES cell-derived keratinocytes of claim 18 to a subject in need of such treatment in an amount effective to treat the skin injury.

49. The method of claim 48, wherein the skin injury is the result of disease or trauma.

50. The method of claim 49, wherein the trauma is a burn.
